(19) United States
(12) Patent Application Publication
(10) Pub. No.: US 2021/0290839 A1
(43) Pub. Date: Sep. 23, 2021

(54) SYSTEMS AND METHODS FOR DETECTING AND TREATING BACTERIAL BIOFILMS

(71) Applicant: UNIVERSITY OF MARYLAND, COLLEGE PARK, College Park, MD (US)

(72) Inventors: Ryan HUISZOON, Silver Spring, MD (US); Pradeep Ramiah RAJASEKARAN, Hyattsville, MD (US); Reza GHODSSI, Potomac, MD (US)

(21) Appl. No.: 17/330,753

(22) Filed: May 26, 2021

Related U.S. Application Data
(62) Division of application No. 16/107,869, filed on Aug. 21, 2018.
(60) Provisional application No. 62/594,478, filed on Dec. 4, 2017.
(60) Provisional application No. 62/548,515, filed on Aug. 22, 2017.

(51) Int. Cl. A61M 5/00; A61M 5/142; A61B 5/00; A61B 5/20; A61B 5/145

(52) U.S. Cl. CPC A61M 5/001; A61M 5/142; A61B 5/6852; A61M 25/0017; A61B 5/14507; A61B 5/207; A61B 5/20

(57) ABSTRACT

Impedance is detected between electrodes coupled to an indwelling medical device to monitor bacteria growth and/or formation of a bacterial biofilm in an in vivo environment. When an antimicrobial agent is present in the in vivo environment, the same voltage that enables impedance detection also generates a bioelectric effect, which decreases a size of the bacterial biofilm or inhibits growth of the bacterial biofilm. In some embodiments, the impedance detected between the electrodes can also be used as a feedback mechanism. When detection indicates that a bacterial biofilm has formed, the system can take remedial measures, such as introducing an antimicrobial agent to the in vivo environment and/or delivering a voltage higher than detecting voltage to deliver a greater bioelectric effect. In some embodiments, the electrodes can be formed on a flexible substrate that is mounted on and conforms to a non-flat surface of the indwelling medical device.

### CROSS-REFERENCE TO RELATED APPLICATIONS

The present application claims the benefit of U.S. Provisional Application No. 62/548,515, filed Aug. 22, 2017, and U.S. Provisional Application No. 62/594,478, filed Dec. 4, 2017, which are hereby incorporated by reference herein in their entireties.

### FIELD

The present disclosure relates generally to detection of bacterial biofilms, and more particularly, to electrical detection that also can be used to treat bacterial biofilms.

### SUMMARY

Embodiments of the disclosed subject matter can monitor bacteria growth and/or formation of a bacterial biofilm in real-time in an in vivo environment. A device with electrodes can be mounted on an indwelling medical device, which is inserted into the in vivo environment. Impedance detected between these electrodes can provide a measure of bacteria growth on the indwelling medical device. When an antimicrobial agent is present in the in vivo environment, the same voltage that enables impedance detection can generate a bioelectric effect, which decreases a size of the bacterial biofilm or inhibits growth of the bacterial biofilm.

The impedance detected between the electrodes can also be used as a feedback mechanism. When the detection indicates that a bacterial biofilm has formed, remedial measures can be taken, such as by introducing an antimicrobial agent to the in vivo environment and/or delivering a voltage higher than detecting voltage to generate a greater bioelectric effect. In some embodiments, the electrodes can be formed on a flexible substrate that is mounted on and conforms to a curved, irregular, or otherwise non-flat surface of the indwelling medical device.

In one or more embodiments, a method includes delivering an antimicrobial agent to an in vivo environment in which an indwelling medical device is disposed. The method further includes, during a first time period, measuring a first impedance value by applying a first voltage between electrodes disposed over a surface of the indwelling medical device. The method also includes determining information regarding bacteria growth on the indwelling medical device based on a comparison of the first impedance value with a previously measured impedance value. The application of the first voltage between the electrodes in the presence of the antimicrobial agent generates a bioelectric effect that decreases a size of a biofilm of the bacteria or inhibits growth of the bacterial biofilm.

In one or more embodiments, a system includes an indwelling medical device, electrodes, and a controller. The indwelling medical device is constructed to be disposed within an in vivo environment. The electrodes are disposed over a surface of the indwelling medical device. The controller can be configured to receive a signal indicative of a first impedance value measured during application of a first voltage between the electrodes and to determine information regarding bacteria growth on the indwelling medical device based at least in part on said signal.

Objects and advantages of embodiments of the disclosed subject matter will become apparent from the following description when considered in conjunction with the accompanying drawings.

### BRIEF DESCRIPTION OF DRAWINGS

Embodiments will hereinafter be described with reference to the accompanying drawings, which have not necessarily been drawn to scale. Where applicable, some elements may be simplified or otherwise not illustrated in order to assist in the illustration and description of underlying features. Throughout the figures, like reference numerals denote like elements.

FIG. 1 is a simplified schematic diagram of a system for detecting and treating bacterial biofilms, according to one or more embodiments of the disclosed subject matter.

FIG. 2A is an exemplary process flow diagram for detecting and treating bacterial biofilms, according to one or more embodiments of the disclosed subject matter.

FIG. 2B is another exemplary process flow diagram for detecting and treating bacterial biofilms, according to one or more embodiments of the disclosed subject matter.

FIGS. 3A-3B are planar and cross-sectional views, respectively, of an exemplary device for detecting and treating bacterial biofilms, according to one or more embodiments of the disclosed subject matter.

FIGS. 4A-4C show different stages of bacterial biofilm growth on the exemplary device of FIG. 3A.

FIG. 5A is a graph of average real-time impedance sensing results over a 24-hour growth period, measured using a benchtop potentiostat with a device fabricated based on the design of FIG. 3A.

FIG. 5B is a graph of average real-time impedance sensing results over a 24-hour growth period, measured using an impedance converter with a device fabricated based on the design of FIG. 3A.

FIG. 6A is a graph of average impedance change during a treatment period with antibiotics while measuring the bacterial biofilm using a device fabricated based on the design of FIG. 3A.

FIG. 6B is a bar graph showing the results of a crystal violet absorbance assay biomass quantification for different treatment conditions, including combined electrical measuring and antibiotics.

FIG. 7 is a graph of real-time fractional relative change during a treatment period for different treatment conditions, including combined electrical measuring and antibiotics.

FIG. 8 is a bar graph showing the results of end-point confocal microscopy quantification of bacterial biofilm thickness for different treatment conditions, including combined electrical measuring and quorum sensing inhibitors (e.g., autoinducer-2 analog).

FIG. 9A is a simplified illustration of a system for electrically detecting and treating a bacterial biofilm for a urinary catheter, with enlarged view of the biofilm detecting portion in the inset, according to one or more embodiments of the disclosed subject matter.

FIG. 9B is a cross-sectional view of the detecting portion of the system of FIG. 9A within the urinary catheter, according to one or more embodiments of the disclosed subject matter.

FIG. 9C is a simplified illustration of a process for installing the detecting portion of the system of FIG. 9A within the urinary catheter, according to one or more embodiments of the disclosed subject matter.

FIG. 10 is a simplified cross-sectional view of an alternative disposition of a detecting portion on a curved, irregular, or otherwise non-flat surface of an indwelling medical device, according to one or more embodiments of the disclosed subject matter.

### DETAILED DESCRIPTION

Embodiments of the disclosed subject matter can monitor bacteria growth and/or formation of a bacterial biofilm in real-time in an in vivo environment. In particular, by detecting impedance between electrodes disposed within the in vivo environment, bacteria growth and/or bacterial biofilm formation can be measured. Such real-time in vivo detection can provide sensitive, early detection of bacterial infection before clinical symptoms might otherwise present.

Moreover, when an antimicrobial agent, such as an antibiotic or a quorum sensing inhibitor, is present in the in vivo environment, the same voltage used for impedance detection synergistically enhances the effect of the antimicrobial agent to decrease a size of the bacterial biofilm or inhibit growth of the bacterial biofilm, to a greater degree than the antimicrobial agent alone. This synergistic enhancement, referred to herein as bioelectric effect, may enable a dosage of antimicrobial agent (e.g., less than a minimum inhibitory concentration for an antibiotic) that is less than would otherwise be required to effectively treat the bacterial infection.

Because of the earlier detection offered by the disclosed systems, methods, and devices, the antimicrobial agent can be delivered early in the development of the biofilm, before the biofilm becomes too great in size and thus more resistant to penetration by the antimicrobial agent. The bioelectric effect in combination with the timing of delivery of the antimicrobial agent allows for the use of lower and more targeted dosages, which may help avoid the development of drug-resistant bacterial infections.

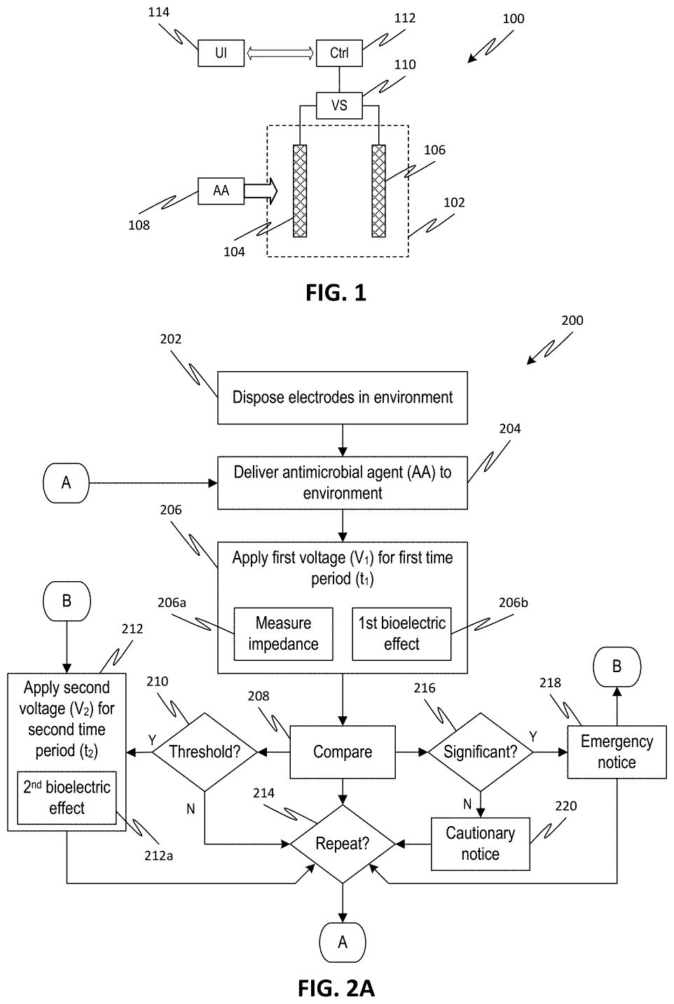

Referring to FIG. 1, a simplified schematic diagram of aspects of a system 100 for detecting and/or treating bacterial biofilms in an in vivo environment 102 is shown. The system 100 can include at least one pair of electrodes 104, 106 disposed within the in vivo environment 102. For example, the electrodes 104, 106 can be disposed on a substrate mounted on a surface of an indwelling medical device, such as a catheter, a needle, or an implant. When the indwelling medical device has a curved, irregular, or otherwise non-flat surface (e.g., the inner surface of the lumen of a catheter), the substrate may be a flexible substrate that conforms to that surface, thereby minimizing or at least reducing obstruction of the indwelling medical device or in the in vivo environment, as well as reducing the number of surfaces available for bacteria growth.

Since the electrodes 104, 106 are to be disposed within an in vivo environment 100, the electrodes 104, 106, and any substrate upon which the electrodes are formed, can be made of bio-compatible materials. For example, the electrodes can be formed of gold. At least the surface of the substrate upon which the electrodes are formed may be substantially insulating. For example, the surface of the substrate, or the entire substrate, can be formed of polyimide when the substrate requires flexibility. In another example, the surface of the substrate, or the entire substrate, can be formed of glass, silicon, or silicon dioxide.

Electrode configurations and quantities other than those illustrated in FIG. 1 are also possible according to one or more contemplated embodiments. For example, electrodes 104, 106 may be configured as a meandering pattern rather than straight lines. Alternatively or additionally, more than a single pair of electrodes can be used, such as in an interdigitated electrode (IDE) pattern. Any electrode configuration where the electrodes are spatially separated from each other and capable of measuring impedance are thus possible and within the scope of the disclosed subject matter.

A voltage source 110 is electrically connected to the electrodes 104, 106 to apply a voltage therebetween. For example, the voltage source 110 can apply an AC voltage, or an AC voltage with a DC offset, between the electrodes 104, 106. The voltage source 110 can also be configured to measure an impedance between the electrodes 104, 106. For example, the voltage source 110 can be a potentiostat or an impedance converter, although any device capable of providing the desired voltage and measuring impedance between the electrodes is also possible according to one or more contemplated embodiments.

Controller 112 can control operation of the system 100. In particular, the controller 112 may be operatively connected to the voltage source 110, for example via wired or wireless (e.g., Bluetooth) connection. In response to commands from the controller 112, the voltage source 110 may apply the detection voltage to the electrodes 104, 106 and measure impedance between the electrodes 104, 106. The voltage source 110 can then send a signal to the controller 112 indicative of the measured impedance. In some cases, the voltage source 110 may send a raw signal, which the controller 112 processes to yield the measured impedance value and thus determine a state of bacteria growth. In other cases, the voltage source 110 may process the raw data before sending to the controller 112, such that the controller 112 receives a signal specifying the measured impedance value or a state of the bacteria growth. The controller 112 may control the voltage source 110 to perform the measurement periodically, continuously, or on-demand (e.g., via a request by a patient, medical professional, or other user through user interface 114).

The determined state of bacteria growth, whether performed by the voltage source 110 or the controller 112, may involve a comparison of the measured impedance value with a previously measured impedance value, where decreases in impedance value reflect increased bacteria growth and/or biofilm formation. Thus, the trend of impedance values over time can provide an indication of relative changes in bacteria within the in vivo environment. Alternatively or additionally, the determined state of bacteria growth may involve a comparison with a predetermined range (e.g., whether the current measured impedance value is below a predetermined threshold).

The controller 112 can communicate with a user interface 114, for example, via wired or wireless (e.g., Bluetooth) connection. For example, the user interface 114 can be an app running on a smartphone, a standalone unit separate from the controller 112, or integrated with the controller 112. The user interface 114 can display information regarding bacteria growth and/or biofilm formation to a patient, medical professional, or other user. For example, when the controller 112 detects decreasing impedance values over time indicative of growth of bacteria, the controller 112 may send an alert or notification to be displayed on the user interface 114, which may be accompanied by visual or auditory alarms. In some embodiments, the controller 112 may base the alert or notification on whether the impedance value is outside of a predetermined range (e.g., below a threshold impedance value corresponding to unacceptable biofilm formation) and/or whether the change in impedance values over time is statistically significant.

In response to the alert or notification on the user interface 114, the user can administer an antimicrobial agent 108 to the in vivo environment 102, for example, by injection, infusion, or ingestion, to treat the bacterial infection. Alternatively or additionally, administration of the antimicrobial agent 108 can be controlled by the controller 112, for example, by sending a signal to a syringe pump, an infusion pump, an implanted drug delivery device, or any other delivery device to deliver the antimicrobial agent.

The antimicrobial agent can be an antibiotic, a quorum sensing inhibitor (e.g., autoinducer-2 analog), or a combination thereof. Autoinducer-2 is a class of small molecules produced by a variety of species of bacteria that mediate communication among various bacteria, including those of disparate genetic history. Analogs of the autoinducer-2 molecules work through the native signal transduction pathway and block signaling, inhibiting quorum sensing. In other words, autoinducer-2 analog molecules, upon uptake by the bacteria, bind to and prevent transcription of genes crucial to quorum sensing, thereby inhibiting or reducing biofilm formation. As is known in the art, synthetic autoinducer-2 analogs can be engineered to target different species of bacteria by changing the alkyl group attached to Cl carbon.

As noted above, the voltage applied between the electrodes 104, 106 during the impedance sensing generates a bioelectric effect, whereby the voltage synergistically enhances the antimicrobial agent 108 to increase the efficacy thereof. As a result of the bioelectric effect, a lower dosage for the antimicrobial agent 108 can be used to treat the bacterial biofilm than would otherwise be required. For example, antibiotics at a dosage at or below the minimum inhibitory concentration (MIC) for the bacteria can be used with the impedance sensing voltage to treat the infection. Thus, the system 100 can provide simultaneous monitoring and treatment of bacterial biofilms within the in vivo environments in real-time.

In some embodiments, in response to the alert or notification on the user interface 114 (or otherwise, when the measured impedance falls outside a predetermined range), the controller 112 can initiate a second bioelectric effect. In particular, the voltage source 110 can apply a second voltage between the electrodes 104, 106, where the second voltage is greater (i.e., has a greater amplitude) than the voltage used for the impedance detection. For example, the second voltage may be at least three times (e.g., ten times) greater than that used for impedance detection. Thus, impedance detection of biofilm growth using electrodes 104, 106 can be used to provide feedback for triggering of a greater bioelectric treatment, by applying a higher voltage via the same electrodes. Such a closed-loop feedback, when coupled with appropriate selection of treatment parameters (i.e., threshold, voltage level, and/or application period), can minimize the amount of time during which the higher voltage is applied. For example, the higher second voltage may be applied for only a small fraction of the total treatment period (e.g., less than 20%).

The user interface 114 can also allow commands to be input to the system 100 by the user. For example, a user can request via the user interface 114 an on-demand impedance reading, by sending a command to voltage source 110 through controller 112. Alternatively or additionally, a user can request the increased-efficacy second bioelectric effect (as described above) on-demand, by sending a command to voltage source 110 to provide the second voltage to the electrodes 104, 106 rather than the normal impedance detection voltage.

Although shown as separate components of system 100 in FIG. 1, it is possible that the illustrated components (or aspects thereof) may be combined together. For example, the user interface 114 and the controller 112 may be combined together as a single component. In another example, the controller 112 and the voltage source 110 may be combined together as a single component. In another example, the user interface 114 and the antimicrobial agent delivery device 108 can be combined together as a single component. In still another example, all components external to the in vivo environment, i.e., the voltage source 110, the controller 112, the user interface 114, and the antimicrobial agent delivery device 108, can be combined together as a single component. Other variations are also possible according to one or more contemplated embodiments.

For example, when the system 100 is intended for use with a urinary catheter, the controller 112 and voltage source 110 may be a microcontroller and impedance converter combined in a single housing proximal to the catheter outside the patient (e.g., on or near a waste container). The electrodes 104, 106 can be disposed within a lumen of the catheter, and the user interface 114 can be a handheld unit (e.g., smartphone) disposed remote from the controller/voltage source housing. Communication between the user interface 114 and the controller 112 may be via a wireless connection (e.g., Bluetooth, Wi-Fi, etc.). Alternatively or additionally, part of the communication between the user interface 114 and the controller 112 may travel over a wired network (e.g., Internet connection). Voltage to the electrodes 104, 106 from the voltage source 110 may be provided via wiring extending along the catheter, e.g., within the lumen of the catheter.

Moreover, other components not illustrated may also be provided as part of system 100, whether integrated with the illustrated components or separate therefrom. For example, the controller 112 can include a memory for storing operating code, measured impedance values, and/or predetermined threshold values, and a processor (e.g., microcontroller) for executing the operating code. Other illustrated components can similarly include additional unillustrated components for effecting operation thereof.

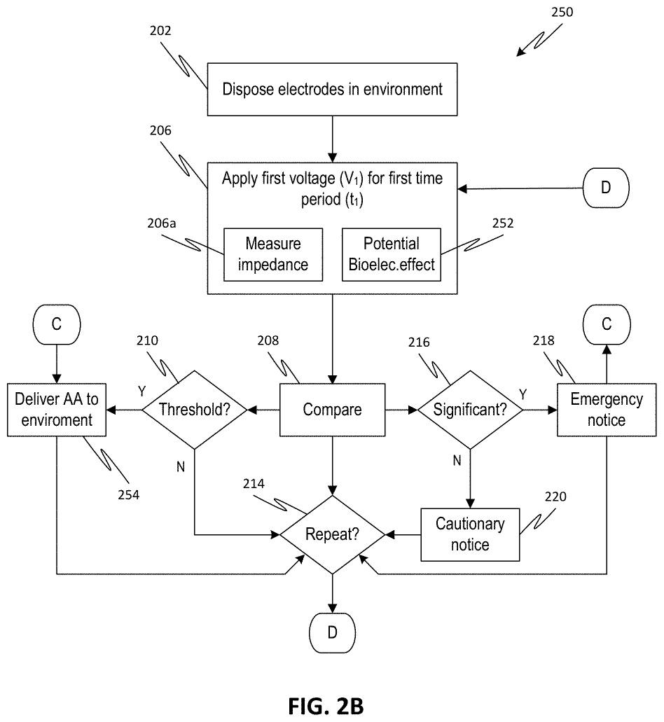

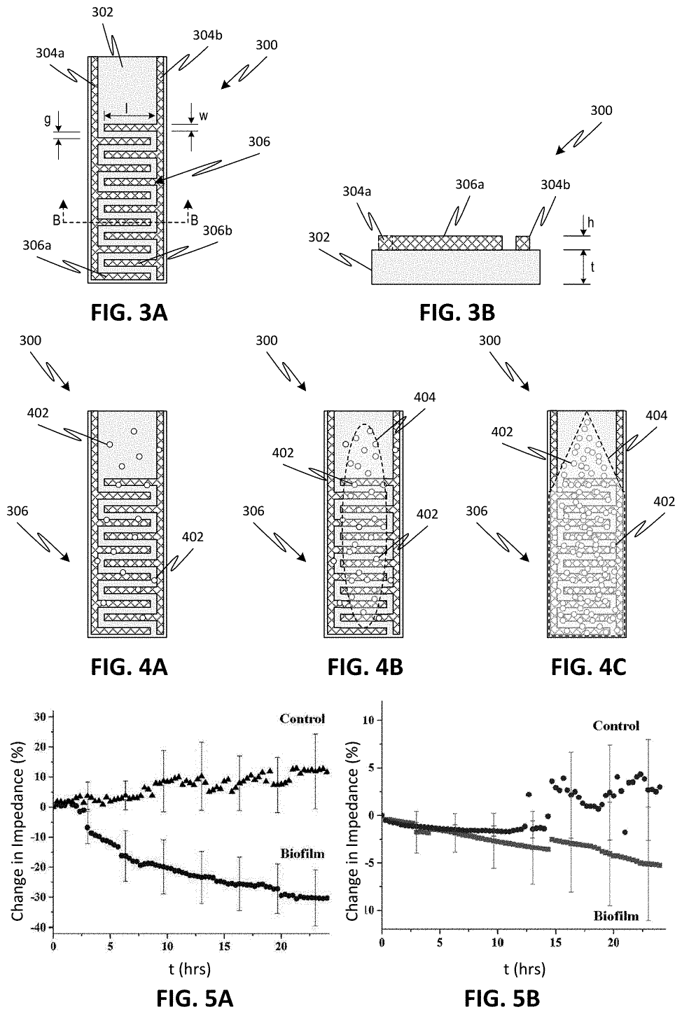

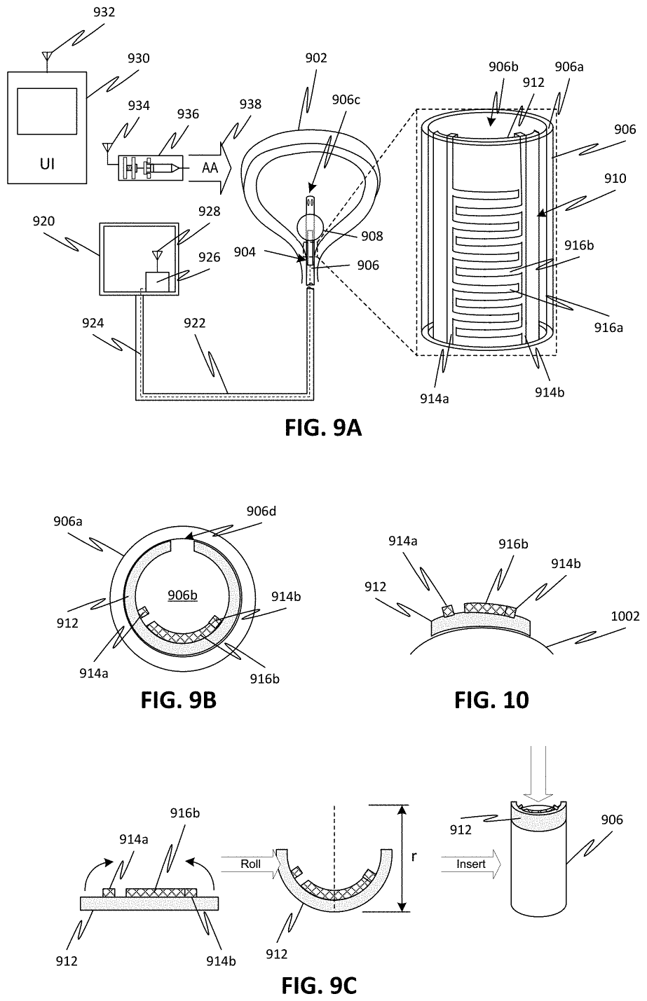

Referring to FIG. 2A, a flow diagram for an exemplary process 200 for detecting and treating bacteria biofilms is illustrated. For example, the process may employ the generalized device illustrated in FIG. 1, the more specific devices illustrated in FIGS. 3A-3B or FIGS. 9A-9B, or variations thereof. The process 200 can include 202, where electrodes are disposed within an in vivo environment. For example, the electrodes can be coupled to an indwelling medical device, which is then inserted or implanted into the in vivo environment of a patient. In some embodiments, the electrodes are disposed on a flexible substrate, which is manipulated and affixed to a non-flat surface of the indwelling medical device, thereby allowing the electrodes to seamlessly integrate with a surface of the indwelling medical device.

The process 200 can further include 204, where an antimicrobial agent is delivered to the in vivo environment. As noted above, the antimicrobial agent can be an antibiotic, a quorum sensing inhibitor (e.g., autoinducer-2 analog), or a combination thereof. The delivery 204 can be by injection, infusion, or ingestion. When antibiotics are used, the dosage may be at a concentration less than or equal to the MIC. Although shown as occurring after 202, it is also possible for the antimicrobial agent delivery 204 to occur prior to the disposing of the electrodes 202, or after the application of impedance sensing voltage 206 (e.g., as in FIG. 2B).

The process 200 can proceed to 206, where a first voltage (V₁) is applied between the electrodes for a first time period (t₁). For example, the first voltage may be an AC voltage signal with an amplitude of 100 mV or less (for example, 50 mV or less, e.g., 5 mV) and a frequency of 2 kHz or less (for example, 100 Hz). The first voltage may be applied intermittently for the first time period, after which no voltage may be applied to the electrodes. During application of the first voltage, the impedance between the electrodes can be measured at 206a. At a same time, the first voltage also enhances the efficacy of any antimicrobial agent present in the in vivo environment, thereby generating a first bioelectric effect 206b. The duration of the first time period and the voltage-free interval between consecutive first time periods may be selected to ensure a desired efficacy for the first bioelectric effect 206b. For example, the total cycle time may be 180 s or less (e.g., 150 s), with the first time period being about 30 s of the total cycle time and the remainder (e.g., 120s) being a voltage-free interval.

The process 200 can proceed to 208, where the detected impedance is compared. For example, the detected impedance can be compared to a predetermined range at 210. If the detected impedance is outside of the range (e.g., impedance is below a threshold previously determined to correspond with formation of a bacterial biofilm), then the process can proceed to 212 where a second voltage (V₂) is applied to the same electrodes for a second time period (t₂). For example, the second time period could be in the normally voltage-free intervals between consecutive first time periods, or in place of one of the first time periods.

In general, the second voltage is greater than the first voltage but less than a voltage that would cause electrolysis of water (e.g., an electric field less than 1.25 V/cm), so as to initiate a second bioelectric effect 212a that has a greater effect against the bacteria biofilm than the first bioelectric effect 206b. For example, the second voltage can be an AC signal that has an amplitude at least three times that of the first voltage (e.g., at least 10 times, or at least 20 times). The AC signal for the second voltage can have a frequency on the order of that of the first voltage (e.g., both at 100 Hz) or at a frequency higher than that of the first voltage (e.g., 1 MHz when the first voltage is at 100 Hz). After application of the second voltage at 212 or if the detected impedance is within range at 210, the process can repeat at 214, for example, by returning to antimicrobial agent delivery 204 (as shown) or to application of the first voltage at the appropriate first time period 206 (not shown).

Alternatively or additionally, the detected impedance can be compared at 208 with one or more previously measured impedance values. Decrease of the current impedance value as compared to the previous impedance values may be indicative of an increase in size of the bacteria cultures and/or the formation/growth of bacterial biofilm. If the comparison at 208 indicates that the currently measured impedance value has decreased with respect to the previously measured value(s), it can be ascertained at 216 whether such change is statistically significant.

If the change is statistically significant, an emergency notification 218 may be sent to the patient, a medical professional, or other user. For example, in response to the emergency notification 218, one or more remedial measures can be taken, such as initiating the second bioelectric effect 212a, increasing dosage of antimicrobial agent, changing type of antimicrobial agent, and/or introducing antimicrobial agent (i.e., see 254 in FIG. 2B). If the change is not statistically significant, a cautionary notification 220 may be sent, in which case the patient, medical professional, or other user may adopt more vigilant monitoring. For example, the duration of the voltage-free intervals may be decreased, or the duration of the first time periods increased, to allow for more frequent impedance measurements (and concurrent bioelectric effect) to more closely monitor progression of the potential bacterial infection. Alternatively, if the change at 216 is not statistically significant, no notification or only an internal system notification, which is not otherwise communicated to the user, may be provided. After the emergency notification 218 or the cautionary notification 220, the process can repeat at 214, for example, by returning to antimicrobial agent delivery 204 (as shown) or to application of the first voltage at the appropriate first time period 206 (not shown).

In some embodiments of the disclosed subject matter, in vivo monitoring of bacteria growth and biofilm formation may occur prior to introduction of any antimicrobial agent. The antimicrobial agent may thus be introduced in response to positive detection of bacteria growth, thereby avoiding the unnecessary use of antimicrobial agents. FIG. 2B illustrates such an exemplary process 250. Similar to process 200 of FIG. 2A, process 250 of FIG. 2B includes disposing the electrodes in the in vivo environment 202 and applying a first voltage 206 to measure impedance 206a. However, since there is no antimicrobial agent initially present in the in vivo environment, there is little or no bioelectric effect.

Moreover, if the comparison 208 indicates that the measured impedance is outside of a predetermined range at 210, the antimicrobial agent can then be introduced to the in vivo environment at 254. Subsequent applications of the first voltage 206, via repeat 214, thus yield a bioelectric effect 252 while simultaneously detecting bacteria growth and/or biofilm formation via the measured impedance 206a.

Although the process flow 250 of FIG. 2B is illustrated separate from the process flow 200 of FIG. 2A, it is also contemplated that the two process flows could be merged together. For example, the process flow 250 of FIG. 2B can operate prior to the introduction of any antimicrobial agent. However, after the introduction of the antimicrobial agent in process flow 250 (for example, at 254), the process flow 200 may take over (for example, where 254 becomes 204 in process flow 200). Other integrations between the two process flows 200, 250 are also possible according to one or more contemplated embodiments.

Referring to FIGS. 3A-3B, an exemplary device 300 for detecting and treating bacterial biofilms is illustrated. The device 300 includes an array 306 of interdigitated electrodes 306a, 306b on a substrate 302. The electrodes 306a, 306b can be traces of a biocompatible conductive material. For example, the electrodes can be formed of gold (with an adhesion layer of 20 nm of chromium between the substrate 302 and the electrodes 306a, 306b) having a height (h) of 200 nm, a width (w) of 300 μm, a length (1) less than 9 mm, and a gap (g) between adjacent electrodes of 300 μm.

Electrodes 306a are connected together at one end thereof by a first lead 304a, while electrodes 306b are connected together at an opposite end thereof by a second lead 304b. Formation of the electrode array 306, as well as the leads 304a, 304b, may be accomplished using standard microfabrication techniques, including, but not limited to material deposition (e.g., evaporation, sputtering, electron beam deposition, etc.), photolithography, wet etching, and dry etching (e.g., reactive ion etching, laser machining, etc.).

The leads 304a, 304b can extend (e.g., at least 30 mm) from the electrode array 306 to remote point on the substrate 302, where voltage from a voltage source (not shown) can be applied for impedance detection (e.g., via contact pads on the substrate 302). The leads 304a, 304b are also formed of a biocompatible conductive material, and may be the same or different than the material of the electrodes 306a, 306b. In some embodiments, the leads 304a, 304b outside of the electrode array 306 can be covered with an insulating substrate. In other embodiments, the leads 304a, 304b are directly exposed to the in vivo environment.

The substrate 302 may be formed of a biocompatible insulating material. In some embodiments, the substrate 302 may be substantially flexible (i.e., capable of being manipulated to have a radius of curvature less than 6 mm without fracture or damage) so as to conform to a surface of an indwelling medical device to which the substrate 302 will be attached. For example, the substrate 302 may be formed of polyimide having a thickness (t) of 25.4 μm. In another example, the substrate 302 may include a silicon oxide layer on a carrier substrate (e.g., silicon substrate) or a glass substrate.

Although FIGS. 3A-3B illustrate a particular number of electrodes 306a, 306b, the number has been chosen for simplicity of illustration, and practical applications of the device 300 may include more than the number of electrodes illustrated. For example, a planar area of the electrode array 306 can be 10 mm×40 mm and would thus include many more than the twelve electrodes illustrated in FIG. 3A. Moreover, although particular dimensions and materials for components of the device 300 have been discussed above, other materials and/or dimensions are also possible according to one or more contemplated embodiments.

The electrode array 306 of device 300 is directly exposed to the in vivo environment such that bacteria can directly form on and between the electrodes of the array 306, thereby changing an impedance between adjacent electrodes 306a, 306b. Thus, during a first time period illustrated in FIG. 4A, individual bacteria colonies 402 form on the device 300 but are insufficient to substantially alter the impedance between the electrodes 306a, 306b, thereby resulting in a relatively high measured impedance. Bacteria colonies on the device 300 can continue to grow and aggregate, with an extracellular matrix 404 (i.e., bacterial biofilm) forming therebetween, as illustrated in FIG. 4B. The increased coverage of the biofilm 404 over the electrode array 306 results in a decrease in the impedance as compared to the scenario of FIG. 4A. Further increases in bacteria 402 growth and biofilm 404 formation on the electrode array 306, as illustrated in FIG. 4C, yields further decreases in the impedance.

As the biofilm grows, impedance between electrodes of the array 306 decreases in a frequency-dependent manner, which decrease may be attributed to changes in double-layer capacitance under 5 kHz. For example, a frequency of 100 Hz may be used for the detecting voltage between electrodes in order to maximize, or at least increase, signal-to-noise ratio. Thus, decreases in the measured impedance can be correlated with formation and growth of bacterial biofilm.

Note that the bacteria growth and biofilm progression illustrated in FIGS. 4A-4C is intended to show the effect of bacteria growth on impedance measurement by the electrode array when no antimicrobial agent is present. In the presence of an antimicrobial agent, especially when combined with the application of the detection voltage to the electrode array, growth of the biofilm would be inhibited or reversed, as discussed in further detail below.

FIG. 5A illustrates the measured impedance characteristics from an experiment employing a detecting device fabricated according to the structure of FIGS. 3A-3B and employing a benchtop potentiostat as the voltage source. FIG. 5B illustrates the measured impedance characteristics from an experiment similar to FIG. 5A, but employing an impedance converter (e.g., AD5933 impedance converter) as the voltage source instead of a potentiostat.

In particular, each experiment employed a flow system including a growth media reservoir, waste reservoir, and the detecting device connected using polymer tubing and luer connectors in between the reservoirs. Fluid was pumped through the flow system by a peristaltic pump. The detecting device and tubing were placed in an incubator maintained at 37° C., to mimic the temperature found in an inserted catheter. Escherichia coli (K12 W3110) were cultured in incubator at 37° C. and then diluted to an OD600 of 0.25. 1 ml of the diluted E. coli solution was then introduced into the catheter tube directly via syringe. The bacteria were allowed to attach to the interior surface under static (no flow) conditions for 2 hours (referred to as the Seeding phase). Immediately following Seeding, pure Luria broth (LB) media was flowed at a constant flow rate of 7 ml/h for 24 hours (referred to as the Growth phase). Throughout the Growth phase, the system impedance (Z) is measured by the detecting device every 2 minutes, with the relative change being used to monitor biofilm formation. Devices without any bacterial cells introduced were also tested as a control (“Control” in FIG. 5A).

As shown in FIG. 5A, the signals for both samples show relatively little change in the first two hours. However, from hours two to three, the impedance of the biofilm sample (“Biofilm” in FIG. 5A) abruptly decreases by 7.2%, likely due to the rapid proliferation phase of biofilm growth. Over the course of the entire Growth phase the system impedance exhibits a dramatic 30% decrease. The impedance decrease is due to the accumulation on the sensor surface of the detecting device of charged proteins and ions associated with the biofilm and biofilm metabolism, which causes a shift in the double layer capacitance. Crystal violet (CV) absorbance assays confirm the correlation of impedance to biofilm growth, the details of which experiments can be found in the underlying provisional applications. By comparison, the control sample exhibits a steady increase of about 10% over the entire growth phase, which increase is due to the formation of air bubbles as air diffuses through the tube.

Similar results are illustrated in FIG. 5B, where the impedance converter measured a gradual decrease of approximately 5% after 24 hours of biofilm growth (“Biofilm” in FIG. 5B). The biofilm-free samples (“Control” in FIG. 5B), by contrast, showed a slight increase of about 2% in impedance after 24 hours. Although FIG. 5B suggests a decreased sensitivity when employing an impedance converter as the voltage source, the differences are believed to be due to how each system is calibrated. Further improvements in sensitivity of a system employing the impedance converter can be achieved by providing additional electronic circuitry for signal processing.

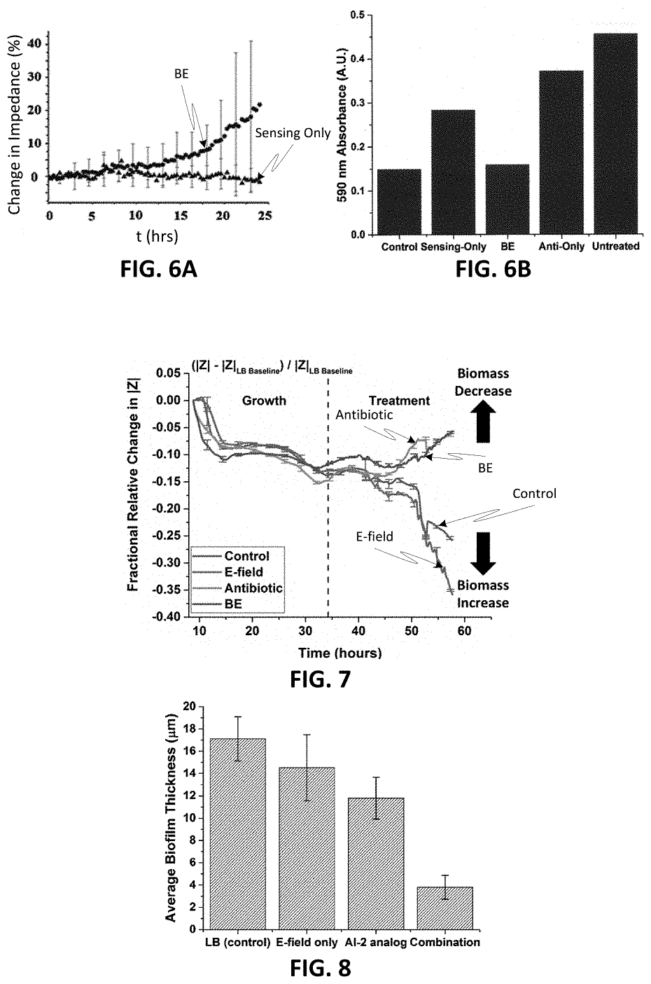

As noted above, the sensing voltage applied to the electrodes of the detecting device 300 can also synergistically enhances any antimicrobial agent present to deliver a bioelectric effect. FIG. 6A illustrates the measured impedance characteristics in experiments employing a detecting device fabricated according to the structure of FIGS. 3A-3B and employing a benchtop potentiostat as the voltage source.

In the experiments, a biofilm was first grown in a cylindrical environment (i.e., growth phase) for 24 hours, and then subjected (i.e., in a treatment phase) to either sensing without any antibiotic present (i.e., “Sensing Only” in FIG. 6A) or sensing with a broad spectrum antibiotic, gentamicin, at a concentration of 10 μg/ml (i.e., “BE” in FIG. 6A) for 24 hours. Impedance measurements were taken by the potentiostat applying an AC voltage of 50 mV at 100 Hz. In order to induce the electric field-based bioelectric effect treatment, this signal was applied at 150 s intervals, and each measurement lasted approximately 30 s of this interval (thus having an intervening interval without voltage application of approximately 120 s).

FIG. 6A shows 50 mV impedance transients at 100 Hz over the course of the 24-hour treatment period for the synergistic bioelectric treatment (“BE” in FIG. 6A) and the untreated control (“Sensing Only” in FIG. 6A). The impedance increased ˜20% during the treatment period with the synergistic treatment, corresponding to the removal of biofilm. By contrast, the untreated control had a decrease in impedance of ˜1% on average. The large error bars in these experiments are present due to the highly variable stochastic nature of biofilm growth.

CV absorbance staining was used to quantify the end-point biomass and to validate the impedance measurements of FIG. 6A. This process involved flowing the CV stain into the tube and allowing it to bind to the adhered proteins and DNA associated with the biofilm. The bound stain is proportional to biofilm biomass. Then, the sample is rinsed with deionized water to remove unbound stain. Decomplexation solution (80% ethanol: 20% acetone) is introduced into the system for 30 minutes. The stain is soluble in decomplexation solution and dissolves into the solution. Optical absorbance at a wavelength (λ) of 590 nm was then measured, with increases corresponding to increased biomass. One-way analysis of variance (ANOVA) was used to determine statistical significance of the biomass quantification results.

FIG. 6B illustrates the results of the CV absorbance assay for various treatment conditions in experiments employing a detecting device fabricated according to the structure of FIGS. 3A-3B and employing a benchtop potentiostat as the voltage source. As illustrated, the bioelectric treatment (i.e., sensing+antibiotic) (“BE” in FIG. 6B) shows biomass similar to a bacteria-free control (“Control” in FIG. 6B). In contrast, sensing-only (i.e., no antibiotic) (“Sensing-only” in FIG. 6B) and antibiotic-only (i.e., no impedance sensing using the detecting device) (“Anti-only” in FIG. 6B) both had significantly higher biomass of biofilm at the end of experiments.

FIG. 7 illustrates the fractional relative change of impedance measurements in experiments similar to that of FIG. 6B. In particular, treatment is performed by applying a 100 mV signal across the same electrodes used for impedance detection, in combination with near MIC levels of antibiotic, thereby generating a second bioelectric effect. This bioelectric effect was applied at regular intervals for only ˜1/7 of the total 24 hour treatment period.

The “E-field” curve represents an experiment where an intervallic 100 mV AC electrical signal (e.g., at 1 MHz) in addition to the periodic 5 mV AC detection voltage was applied to the electrodes, but without any antibiotic present. The “BE” curve represents an experiment where an intervallic 100 mV AC electrical signal (e.g., at 1 MHz) in addition to the periodic 5 mV AC detection voltage was applied to the electrodes, and with 10 μg/mL of gentamicin present. The decision to apply the 100 mV AC signal in both experiments was made by logic using the impedance data gathered in the sensing mode (i.e., using the 5 mV AC detection voltage). If the average sensing mode data point was less than a predefined threshold, then the larger 100 mV electric field treatment was applied; otherwise, the system continued in sensing mode with the periodic application of the 5 mV AC detection voltage. The “Antibiotic” curve represents an experiment where periodic 5 mV AC detection voltage was applied to the electrodes, and with 10 μg/mL of gentamicin present. The “Control” curve represents an experiment where only the periodic 5 mV AC detection was applied to the electrodes.

As illustrated in FIG. 7, at the end of treatment phase, the untreated “Control” and the “E-field” only experiments showed a further decrease in impedance, suggestive of an increase in total biomass or additional biofilm growth. Conversely, treatment with “Antibiotic” and “BE” experiments resulted in an increase in 100 Hz impedance, representing the removal or decrease in total biomass.

Moreover, the “Antibiotic” treatment of FIG. 7 appears to be as effective in treating the biofilm as the “BE” treatment of FIG. 7. This is due to the first bioelectric effect, caused by the lower detection voltage of the periodic impedance sensing applied during the antibiotic only treatment. As a result, the efficacy of the “Antibiotic” treatment of FIG. 7 is not purely a result of the antibiotic alone, but rather due to regular and recurring bioelectric treatment from the periodic sensing that results in effective removal of the biofilm.

Thus, in embodiments, the detection and treatment system can operate in at least two bioelectric effect regimes. In a first regime, the detection voltage (i.e., relatively low voltage, such as 5 mV AC at 100 Hz) applied to the electrodes results in enhanced efficacy of the antimicrobial agent concurrently with the impedance measurement. In a second regime, the system can switch from the lower detection voltage to a higher electric field (e.g., 100 mV AC at 1 MHz) when the detected biofilm growth is outside the bounds of a predetermined range (e.g., when the most recent impedance measurement is more negative than a previously defined threshold). This second regime could be used to help treat significantly thicker and more mature biofilms, which require stronger electrical energy for effective treatment. Such a feedback-based method of treatment with the bioelectric effect at the onset of biofilm formation can avoid, or at least reduce the risk of, forming thick biofilms.

Although the preceding paragraphs address the bioelectric effect based on antibiotics, similar results can be obtained with other antimicrobial agents, such as quorum sensing inhibitors. In particular, FIG. 8 illustrates the results of experiments for different treatment conditions using autoinducer-2 analog. “LB control” represents an experiment where no electric field or autoinducer-2 analog was present. “E-field only” represents an experiment where an electrical signal of 0.125V AC at 10 MHz offset by 0.125V DC was applied to electrodes, without any autoinducer-2 analog present. “AI-2 analog” represents an experiment where autoinducer-2 analog is introduced (e.g., 100 μM autoinducer-2 analog isobutyl DPD) without any electrical signal applied to the electrodes. “Combination” represents an experiment where the electrical signal is applied to the electrodes and autoinducer-2 analog is introduced. As is readily apparent from FIG. 8, the combination of autoinducer-2 analog and voltage application to the electrodes results in substantially improved efficacy in reducing biofilm thickness over either alone, as a result of the bioelectric effect.

Further details regarding the experimental setup and the corresponding results for FIGS. 6A-8 can be found in one or more of the underlying provisional applications, which are incorporated by reference herein.

FIGS. 9A-9C illustrate the detection and treatment aspects of the disclosed subject matter as practically applied to a urinary catheter 906. The urinary catheter 906 has a conventional configuration, with a retention balloon 902 that sits within a patient's bladder 908 to prevent accidental removal. The catheter 906 also has tubing with an inner volume 906b defined by sidewall 906a that extends through the patient's urethra 904 into the bladder 902 and terminates at end 906c with fluidic inlets/outlets. A waste tube 922 can convey waste fluid (i.e., urine) from the terminating end 906c via inner volume 906b to a waste container 920.

A detection and treatment device 910 includes an array of interdigitated electrodes 916a, 916b formed on a flexible substrate 912, which may be disposed on an inner surface of catheter sidewall 906a within the bladder 902 and/or urethra 904. Electrical wiring 924 can convey the detection voltage (or when an enhanced bioelectric effect is desired, a higher second voltage) from controller 926 (which includes a voltage source, such as an impedance converter, and corresponding electronics for control of the system) to the electrodes 916a, 916b via respective leads 914a, 914b. Thus, bacterial biofilms within the bladder environment that form on surfaces of the urinary catheter 906 can be readily detected by the electrode array of the device 910.

Controller 926 is disposed proximal to or on the waste container 920 and can include a wireless transceiver 928 for communicating with a user interface 930, which may be remote from the waste container 920 and/or the patient. For example, the waste container 920 may be mounted on the patient, so that the patient is able to freely move with the urinary catheter in place. The controller 926 may be similarly mounted to the patient, with appropriate wiring and power source for powering the detection device 910 disposed within the catheter 906.

The user interface 930 can be a smart phone or other handheld unit that communicates with the controller 926 via wireless transceiver 932. Alternatively, the user interface 930 can be a standalone unit, for example, when the patient is currently bedridden. In yet another alternative, the user interface 930 may be a remote medical station monitored by a medical professional. In still another alternative, the user interface 930 and the controller 926 can be integrated into a single device carried with or disposed proximal to the patient.

As with other embodiments described above, the user interface 930 and/or the controller 926 can be used to monitor biofilm formation in real-time and to initiate treatments. For example, the user interface 930 or the controller 926 can initiate introduction of an antimicrobial agent 938 in response to detected biofilms (whether automatically by using logic to evaluate or in response to a manual request by a user), such as by sending appropriate commands to a syringe or infusion pump 936 via wireless transceiver 934. The detecting voltage between the electrodes 916a, 916b in the presence of the introduced antimicrobial agent 938 thus provides simultaneous detection and treatment of the bacterial biofilm. Moreover, the user interface 930 or the controller 926 can provide an enhanced bioelectric effect by changing the voltage (i.e., increasing magnitude and/or frequency) applied to the electrodes 916a, 916b.

The flexible substrate 912 can be formed separate from the catheter 906 and then installed within inner volume 906b prior to insertion of the catheter 906 into the patient. In particular, the flexible substrate 912 may be installed on the inner surface of catheter sidewall 906a so as to conform closely to the sidewall profile, as shown in FIG. 9B, thereby reducing the risk of obstructing the inner volume 906b and minimizing the surface area for bacterial biofilm adhesion. The flexible substrate 912 can be rolled, bent, or otherwise non-destructively manipulated to have a radius of curvature (r) that matches that of the urinary catheter into which it is inserted. For example, the flexible substrate 912 is rolled to have a radius of curvature less than 6 mm (e.g., 2.25 mm) and is then slid into the interior volume 906b of the catheter 906, as illustrated in FIGS. 9B-9C. After insertion, the substrate 912 can be attached to the catheter sidewall by various attachment means, such as, but not limited to, glue, epoxy, or curable polymer (e.g., polydimethylsiloxane (PDMS)) disposed between the substrate 912 and the sidewall 906a.

The detection device 910 thus integrated with the catheter 906 allows for detection of bacteria growth and biofilm formation, and simultaneous treatment to inhibit bacteria growth and biofilm formation when an antimicrobial agent is present. The detection device 910 also enables real-time detection for more timely undertaking of remedial measures, such as introduction of antimicrobial agents before a thick biofilm is formed or increasing voltage between the electrodes 916a, 916b for an enhanced bioelectric effect. The timeliness of treatment (i.e., before a thick biofilm forms) coupled with the increased efficacy of the bioelectric effect allows for a lower dosage of the antimicrobial agent (e.g., at or less than MIC) than would otherwise be required to combat the bacterial infection.

Although the specific application of the detecting and treatment of bacterial biofilm in the context of a urinary catheter has been discussed above, embodiments of the disclosed subject matter are not limited thereto. Indeed, the detecting and treatment via electrodes can be applied to other indwelling medical devices as well, such as, but not limited to, coronary catheters, central venous catheters, Quinton catheters, or any other type of medical catheter; hypodermic needles, Tuohy needles, or any other type of medical needle; dental implants, orthopedic implants, coronary/heart valves, or any other type of medical implant. As with the urinary catheter example, the electrodes can be disposed over an internal wall of the lumen of the indwelling medical device. Alternatively or additionally, the electrodes can be disposed over an exterior non-flat surface of the indwelling medical device. FIG. 10 illustrates such an exemplary configuration, where the substrate 912 of the detection device 910 is coupled and conforms to the curved external surface 1002 of a generic indwelling medical device.

Although reference has been made herein to detecting and treating bacterial biofilm in a patient, embodiments of the disclosed subject matter are not limited to use in a human. Indeed, embodiments of the disclosed subject matter can find wide application to non-human in vivo environments (e.g., animal) or any other environment where monitoring and/or treating bacterial growth may be desirable (e.g., benchtop testing setups for studying biofilm growth).

It will be appreciated that the aspects of the disclosed subject matter can be implemented, fully or partially, in hardware, hardware programmed by software, software instruction stored on a computer readable medium (e.g., a non-transitory computer readable medium), or any combination of the above.

For example, components of the disclosed subject matter, including components such as a controller, processor, or any other feature, can include, but are not limited to, a personal computer or workstation or other such computing system that includes a processor, microprocessor, microcontroller device, or is comprised of control logic including integrated circuits such as, for example, an application specific integrated circuit (ASIC).

Features discussed herein can be performed on a single or distributed processor (single and/or multi-core), by components distributed across multiple computers or systems, or by components co-located in a single processor or system. For example, aspects of the disclosed subject matter can be implemented via a programmed general purpose computer, an integrated circuit device (e.g., ASIC), a digital signal processor (DSP), an electronic device programmed with microcode (e.g., a microprocessor or microcontroller), a hard-wired electronic or logic circuit, a programmable logic circuit (e.g., programmable logic device (PLD), programmable logic array (PLA), field-programmable gate array (FPGA), programmable array logic (PAL)), software stored on a computer-readable medium or signal, an optical computing device, a networked system of electronic and/or optical devices, a special purpose computing device, a semiconductor chip, a software module or object stored on a computer-readable medium or signal.

When implemented in software, functions may be stored on or transmitted over as one or more instructions or code on a computer-readable medium. The steps of a method or algorithm disclosed herein may be embodied in a processor-executable software module, which may reside on a computer-readable medium. Instructions can be compiled from source code instructions provided in accordance with a programming language. The sequence of programmed instructions and data associated therewith can be stored in a computer-readable medium (e.g., a non-transitory computer readable medium), such as a computer memory or storage device, which can be any suitable memory apparatus, such as, but not limited to read-only memory (ROM), programmable read-only memory (PROM), electrically erasable programmable read-only memory (EEPROM), random-access memory (RAM), flash memory, disk drive, etc.

As used herein, computer-readable media includes both computer storage media and communication media, including any medium that facilitates transfer of a computer program from one place to another. Thus, a storage media may be any available media that may be accessed by a computer. By way of example, and not limitation, such computer-readable media may comprise RAM, ROM, EEPROM, CD-ROM or other optical disk storage, magnetic disk storage or other magnetic storage devices, or any other medium that may be used to carry or store desired program code in the form of instructions or data structures and that may be accessed by a computer.

Also, any connection is properly termed a computer-readable medium. For example, if the software is transmitted from a website, server, or other remote source using a transmission medium (e.g., coaxial cable, fiber optic cable, twisted pair, digital subscriber line (DSL), or wireless technologies such as infrared, radio, and microwave), then the transmission medium is included in the definition of computer-readable medium. Moreover, the operations of a method or algorithm may reside as one of (or any combination of) or a set of codes and/or instructions on a machine readable medium and/or computer-readable medium, which may be incorporated into a computer program product.

One of ordinary skill in the art will readily appreciate that the above description is not exhaustive, and that aspects of the disclosed subject matter may be implemented other than as specifically disclosed above. Indeed, embodiments of the disclosed subject matter can be implemented in hardware and/or software using any known or later developed systems, structures, devices, and/or software by those of ordinary skill in the applicable art from the functional description provided herein.

In this application, unless specifically stated otherwise, the use of the singular includes the plural, and the separate use of “or” and “and” includes the other, i.e., “and/or.” Furthermore, use of the terms “including” or “having,” as well as other forms such as “includes,” “included,” “has,” or “had,” are intended to have the same effect as “comprising” and thus should not be understood as limiting.

Any range described herein will be understood to include the endpoints and all values between the endpoints. Whenever “substantially,” “approximately,” “essentially,” “near,” or similar language is used in combination with a specific value, variations up to and including 10% of that value are intended, unless explicitly stated otherwise.

The foregoing descriptions apply, in some cases, to examples generated in a laboratory, but these examples can be extended to production techniques. Thus, where quantities and techniques apply to the laboratory examples, they should not be understood as limiting.

The terms “system,” “device,” and “module” have been used interchangeably herein, and the use of one term in the description of an embodiment does not preclude the application of the other terms to that embodiment or any other embodiment.

It is thus apparent that there is provided, in accordance with the present disclosure, systems and methods for detecting and treating bacterial biofilms. Many alternatives, modifications, and variations are enabled by the present disclosure. While specific examples have been shown and described in detail to illustrate the application of the principles of the present invention, it will be understood that the invention may be embodied otherwise without departing from such principles. For example, disclosed features may be combined, rearranged, omitted, etc. to produce additional embodiments, while certain disclosed features may sometimes be used to advantage without a corresponding use of other features. Accordingly, Applicant intends to embrace all such alternative, modifications, equivalents, and variations that are within the spirit and scope of the present invention.

1. A method comprising:
(a) delivering an antimicrobial agent to an in vivo environment in which an indwelling medical device is disposed;
(b) during a first time period, measuring a first impedance value by applying a first AC voltage signal to electrodes disposed over a surface of the indwelling medical device, the first AC voltage signal having a first frequency in a range of 100 Hz to 2 kHz and a first amplitude;
(c) generating an indication of positive bacteria growth on the indwelling medical device in response to the first impedance value being less than a previously measured impedance value; and
(d1) applying a second AC voltage signal to the electrodes in response to the indication of positive bacteria growth or in response to the first impedance value being outside of a first predetermined range,
wherein the applying the first AC voltage signal to the electrodes in the presence of the antimicrobial agent generates a first bioelectric effect, in which efficacy of the antimicrobial agent in decreasing a size of a bacteria culture or biofilm or inhibiting further growth of the bacteria culture or biofilm is synergistically enhanced,
the second AC voltage signal has a second frequency greater than the first frequency, a second amplitude greater than the first amplitude, or both, and
the applying the second AC voltage signal to the electrodes in the presence of the antimicrobial agent generates a second bioelectric effect greater than the first bioelectric effect.

2. (canceled)

3. The method of claim 1, wherein the second amplitude of the second AC voltage signal is at least three times greater than the first amplitude of the first AC voltage signal.

4. The method of claim 1, further comprising:
(d2) comparing the previously measured impedance value to a second predetermined range,
wherein (a) is performed in response to (d2) indicating that the previously measured impedance value is outside said second predetermined range.

5. The method of claim 1, wherein:
the indwelling medical device comprises a catheter, a needle, or an implant;
said surface of the indwelling medical device is a curved, irregular, or non-flat surface;
the electrodes are disposed on a substrate that conforms to a cross-sectional profile of the curved, irregular, or non-flat surface; and
the method further comprises, prior to disposition of the indwelling medical device within the in vivo environment, coupling the substrate to the curved, irregular, or non-flat surface of the indwelling medical device.

6-10. (canceled)

11. The method of claim 1, wherein the antimicrobial agent comprises at least one of an antibiotic and a quorum sensing inhibitor.

12. The method of claim 11, wherein the quorum sensing inhibitor comprises an autoinducer-2 analog.

13. The method of claim 11, wherein a dosage of the antibiotic in (a) is less than a minimum inhibitory concentration for the antibiotic for said bacteria in said in vivo environment.

14. The method of claim 1, wherein no voltage is applied between the electrodes in an interim period after the first time period.

15. The method of claim 14, wherein after the interim period, at least (b) and (c) are repeated.

16. A system comprising:
an indwelling medical device constructed to be disposed within an in vivo environment;
electrodes disposed over a surface of the indwelling medical device;
a voltage source configured to apply AC voltages signals to the electrodes; and
a controller comprising one or more processors and a computer readable storage media storing instructions that, when executed by the one or more processors cause the one or more processors to:
for a first measurement cycle, control the voltage source to apply a first AC voltage signal to the electrodes and receive a first measurement signal indicative of a first impedance value measured during the application, the first AC voltage signal having a first frequency in a range of 100 Hz to 2 kHz and a first amplitude;
for a second measurement cycle after the first measurement cycle, control the voltage source to re-apply the first AC voltage signal to the electrodes and receive a second measurement signal indicative of a second impedance value measured during the re-application;
in response to the second impedance value being less than the first impedance value, generate an indication of positive growth of a bacteria culture or biofilm on the electrodes; and
in response to the indication of positive growth or in response to the second impedance value being outside of a first predetermined range, control the voltage source to apply a second AC voltage signal to the electrodes,
wherein the second AC voltage signal has a second frequency greater than the first frequency, a second amplitude greater than the first amplitude, or both.

17. The system of claim 16, further comprising:
an interface unit disposed external to the in vivo environment and configured to transmit said first and second measurement signals to the controller,
wherein the controller is located remote from and physically unconnected to the indwelling medical device.

18. The system of claim 16, wherein the voltage source is further configured to measure impedance between the electrodes.

19. The system of claim 16, comprising:
a substrate supporting the electrodes thereon,
wherein the substrate is attached and conforms to a curved, irregular, or non-flat surface of the indwelling medical device.

20. (canceled)

21. The system of claim 16, wherein the indwelling medical device comprises a catheter, a needle, or an implant.

22. The system of claim 16, further comprising:
an antimicrobial agent,
wherein application of the first AC voltage signal to the electrodes in the presence of the antimicrobial agent generates a first bioelectric effect, in which efficacy of the antimicrobial agent in decreasing a size of the bacteria culture or biofilm or inhibiting further growth of the bacteria culture or biofilm is synergistically enhanced, and
application of the second AC voltage signal to the electrodes in the presence of the antimicrobial agent generates a second bioelectric effect greater than the first bioelectric effect.

23. The system of claim 22, wherein the antimicrobial agent comprises an antibiotic or a quorum sensing inhibitor.

24. (canceled)

25. The system of claim 16, further comprising:
a syringe or infusion pump for delivering a dose of antimicrobial agent,
wherein the controller is further configured to control the syringe or infusion pump, in response to the indication of positive growth, to administer the dose of antimicrobial agent to the in vivo environment.

26. The system of claim 25, wherein the dose is below a minimum inhibitory concentration of the antimicrobial agent for bacteria of the culture or biofilm.

27. The method of claim 1, wherein the second frequency is at least five-hundred times the first frequency.

28. The method of claim 1, wherein:
in (b), the first AC voltage signal is applied to the electrodes during a first application period of the first time period and no voltage signal is applied to the electrodes during a first remaining period of the first time period following the first application period, a duration of the first remaining period being greater than a duration of the first application period;
the method further comprises, in response to the indication of positive bacteria growth, during a second time period after the first time period, measuring a second impedance value by applying the first AC voltage signal to the electrodes during a second application period of the second time period and applying no voltage signal to the electrodes during a second remaining period of the second time period following the second application period; and
a duration of the second application period is greater than a duration of the first application period, or a duration of the second remaining period is less than a duration of the first remaining period.
